(19) United States
(12) Patent Application Publication
(10) Pub. No.: US 2020/0072842 A1
(43) Pub. Date: Mar. 5, 2020

(54) BLADDER CANCER DETECTION DEVICE AND METHOD

(71) Applicants: University of South Australia, Adelaide, South Australia (AU); Flinders University of South Australia, Bedford Park, South Australia (AU)

(72) Inventors: Krasimir Atanasov Vasilev, Mawson Lakes, South Australia (AU); Melanie Macgregor, Parafield Gardens, South Australia (AU); Jonathan Gleadle, Stirling, South Australia (AU); Jordan Li, Hove, South Australia (AU)

(73) Assignees: University of South Australia, Adelaide, South Australia (AU); Flinders University of South Australia, Bedford Park, South Australia (AU)

(21) Appl. No.: 16/604,990

(22) PCT Filed: Apr. 12, 2018
(86) PCT No.: PCT/AU2018/000053

§ 371 Date: Oct. 11, 2019

(30) Foreign Application Priority Data
Apr. 12, 2017 (AU) 2017901350

(51) Int. Cl. G01N 33/574; G01N 33/569; G01N 33/543

(52) U.S. Cl. CPC G01N 33/57407; G01N 33/54366; G01N 33/56966

(57) ABSTRACT

A device for selective capture of target bladder cancer cells from urine or a urine derived fluid is provided. The device comprises a substrate having one or more cell capture surfaces, each cell capture surface comprising a functionalized film on the substrate and one or more target bladder cancer cell selective binding agents covalently bound to the functionalized film.

### PRIORITY DOCUMENT

The present application claims priority from Australian Provisional Patent Application No. 2017901350 titled “BLADDER CANCER DETECTION DEVICE AND METHOD” and filed on 12 Apr. 2017, the content of which is hereby incorporated by reference in its entirety.

### TECHNICAL FIELD

The present disclosure relates to devices and methods for selectively capturing and/or detecting bladder cancer cells, or biomarkers thereof, from urine. The devices and methods may find particular application in the field of point of care diagnostic devices and methods.

### BACKGROUND

The bladder is the most common location of cancers in the renal tract and the most common histological type is transitional cell carcinoma. Patients with one bladder cancer are at significant risk of other cancers in the renal tract and have particularly high recurrence rates (70%) (Dawam 2012; Youssef and Lotan 27 2011). Therefore, it is necessary for patients with bladder cancer to be monitored regularly and indefinitely.

Currently, bladder cancers are monitored with invasive and costly techniques such as cystoscopy, (Bryan et al. 2014a) which to this day is considered as the gold standard of care by clinicians. Cheaper, less invasive and accurate tests are needed. (Lotan et al. 2009; Ploeg et al. 2009)

One possible non-invasive avenue for the diagnosis of bladder cancer is the analysis of patient urine. Cancer cells of clinically relevant bladder tumors are routinely shed into urine. (Deden 1954; Kiyoshima et al. 2016; McGrew 1961; Murphy 1990) The presence of exfoliated tumor cells in urine is at the basis of cytology examination of voided urine, which is currently the only non-invasive routine standard of care for the surveillance of bladder cancers. (Deden 1954; Gaston and Pruthi 2004; McGrew 1961; Papanicolaou and Marshall 1945; Zhang et al. 2001) Yet, the efficacy of urine cytology is debated, and the sensitivity of this test is unsatisfactorily low (<40%). (Andersson et al. 2014; 40 Mitra and Cote 2010) The reason for the low sensitivity in cytology is the fact that tumor cells, and especially low grade tumor cells, share many similarities with normal cells such as often comparable cytomorphologies. (Brimo et al. 2009; Murphy et al. 1984) Distinguishing between malign and benign cells based on their morphology alone is difficult. For this reason, cytological examination requires experienced medical professionals and time for analysis. (Bryan et al. 2014a)

Another challenge faced by urine cytologists is to obtain a clear field of view of the cell of interest amongst urinary protein, debris and other background cells. (Bastacky et al. 1999) Often, conditions other than cancer induce similar symptoms and lead to increased urinary cellularity, these include but are not limited to infection, or kidney disease. (Oliveira Arcolino 50 et al. 2015) In order to minimise the background, native urine has to be extensively processed, a procedure which increases sample analysis time and cost. (Wronska et al. 2014)

There is thus a need to provide devices and methods capable of selective and sensitive bladder cancer cell capture from urine in order to reduce the reliance on expensive and time consuming methods such as urine cytology and cytoscopic surveillance.

### SUMMARY

The present disclosure arises from our finding that tumour cells shed in the urine of bladder cancer patients can be selectively immuno-captured from unprocessed or native urine and concentrated on a biomaterial platform. Specifically, we found that bladder cancer carcinoma cells can be selectively captured on a substrate surface functionalised with bioactive Epithelial Cell Adhesion molecule (EpCAM) anti-body. EpCAM was chosen as a specific capture ligand because it is the most notorious carcinoma specific antibody. (Bryan et al. 2014b; Patriarca et al. 2012)

In a first aspect, provided herein is a device for selective capture of target bladder cancer cells from urine or a urine derived fluid, the device comprising a substrate having one or more cell capture surfaces, each cell capture surface comprising a functionalized film on the substrate and one or more target bladder cancer cell selective binding agents covalently bound to the functionalized film.

In a second aspect, provided herein is a microfluidic device for selective capture of target bladder cancer cells from urine or a urine derived fluid, the device comprising a substrate having one or more cell capture micro-channels, each cell capture micro-channel comprising a functionalized film on a surface thereof and one or more target bladder cancer cell selective binding agents covalently bound to the functionalized film.

In a third aspect, the present disclosure provides a method of selective capturing target bladder cancer cells from urine or a urine derived fluid, the method comprising:

providing a sample of urine or a urine derived fluid;

providing a substrate having one or more cell capture surfaces, each cell capture surface comprising a functionalized film on the substrate and one or more target bladder cancer cell selective binding agents covalently bound to the functionalized film;

contacting the sample of urine or a urine derived fluid with the one or more cell capture surfaces under conditions to bind at least some of the target bladder cancer cells from the urine (if present) to the cell capture surface.

The method of the third aspect may further comprise detecting target bladder cancer cells on the one or more cell capture surfaces. Captured bladder cancer cells can be detected using a cancer specific fluorescently active compound, such as ALA 5, hexaminolevulinate or hypericin.

In a fourth aspect, the present disclosure provides a method of immobilising target bladder cancer cells from urine or a urine derived fluid on a substrate surface, the method comprising:

providing a sample of urine or a urine derived fluid;

providing a substrate having one or more cell capture surfaces, each cell capture surface comprising a functionalized film on the substrate and one or more target bladder cancer cell selective binding agents covalently bound to the functionalized film;

contacting the sample of urine or a urine derived fluid with the one or more cell capture surfaces under conditions to bind at least some of the target bladder cancer cells from the urine (if present) to the cell capture surface.

In a fifth aspect, the present disclosure provides a method for diagnosing or monitoring bladder cancer in a mammal, the method comprising:

providing a sample of urine or a urine derived fluid obtained from the mammal;

providing a substrate having one or more cell capture surfaces, each cell capture surface comprising a functionalized film on the substrate and one or more target bladder cancer cell selective binding agents covalently bound to the functionalized film;

contacting the sample of urine or a urine derived fluid with the one or more cell capture surfaces under conditions to bind at least some of the target bladder cancer cells from the urine (if present) to the cell capture surface; and

analyzing the target bladder cancer cells bound to the cell capture surface.

In embodiments of the fifth aspect, the step of analyzing the target bladder cancer cells bound to the cell capture surface comprises detecting the target bladder cancer cells using a cancer specific fluorescently active compound. The cancer specific fluorescently active compound may be ALA 5, hexaminolevulinate or hypericin.

### BRIEF DESCRIPTION OF DRAWINGS

Embodiments of the present disclosure will be discussed with reference to the accompanying figures wherein:

FIG. 1 is a schematic of the non-invasive diagnosis approach of the present disclosure;

FIG. 2 summarises results from western blots, immunostaining and FACS analysis confirming the expression of cancer specific EpCAM membrane protein by bladder cancer cell lines;

FIG. 3 shows FACS results identifying EpCAM and E-Cadherin as potential cancer specific membrane markers;

FIG. 4 shows western blots confirming EpCAM expression results obtained by FACS;

FIG. 5 shows immunostaining results confirming the FACS and western blots results;

FIG. 6 shows evidence of the selective capture of bladder cancer cells into functionalised fluidic microchannels, compared to positive and negative control microchannels. Results presented from left to right in terms of number and percentage of cell captured, sensitivity and specificity. The plot on the left shows the number of healthy (F001) and bladder cancer (RT4) cells present on the substrates before (seeding) and after (captured) rinsing the 3 type of microchannels: control=plain PPOx surface, block=blocked PPOx substrate, EpCAN=EpCAM functionalised PPOx substrate. The plot in the middle shows the percentage of cells captured per field of view for at least three tested surfaces. The plot on the right shows the calculated sensitivity and specificity for the examined surfaces from the cancer cell capture experiment;

FIG. 7 shows cell capture sensitivity and selectivity for low cell number spiked in real urine, for two different model bladder cancer cell lines (HDF: left column; HT1376: right column);

FIG. 8 shows grade 3 bladder cancer cells HT1376 captured under the flow conditions from 5 mL of real urine containing only 1000 spiked cells;

FIG. 9 shows cancer cells specific red fluorescence in a co-culture of cancer and healthy cells, and fluorescence spectroscopy results of the fluorescent marker concentration optimisation study for in vitro testing;

FIG. 10 shows an example of the cellularity of a confirmed bladder cancer patient urine sample, with blue nucleus stain and red cancer specific fluorescence;

FIG. 11 shows an example of cancer cell capture results from the urine sample of one patient with confirmed transitional cell carcinoma. Cancer cells contain both blue (nuclear stain) and distinctive red fluorescence (BF: left column; Dapi: middle column; HexAla (Right column);

FIG. 12 shows a negative control example of cancer cell capture results from the urine sample of a healthy patient (BF: left column; Dapi: middle column; HexAla (Right column);

FIG. 13 shows a direct comparison between the cancer cell detection device results and results from cytology and cystoscopy examination of the same samples;

FIG. 14 illustrates the criteria used in the automated counting software to confirm the cancerous nature of cells captured in the microchannels; and

FIG. 15 shows images for the ideal conditions with cancer cell lines HT1376 and HT1197 in 50:50 co-culture with healthy human foreskin fibroblasts (HFF) are shown in the fluorescent micrographs on the right. The top images are an overlay with the bright field image to see the non-fluorescent healthy cells present. In the data plotted on the left of the figure, at each concentration and time point shown the plotted data are from left to right: HFF; HT1197; HT1376; EJ138; RT4 cells.

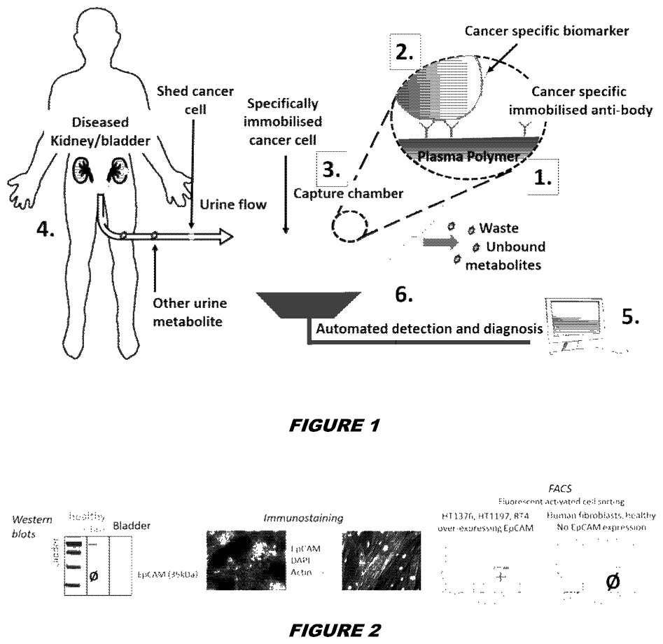

### DESCRIPTION OF EMBODIMENTS

In a first aspect, provided herein is a device for selective capture of target bladder cancer cells from urine or a urine derived fluid, the device comprising a substrate having one or more cell capture surfaces, each cell capture surface comprising a functionalized film on the substrate and one or more target bladder cancer cell selective binding agents covalently bound to the functionalized film.

Advantageously, the device of the first aspect may find use in a point of care device capable of selective bladder cancer cell capture from urine. Over the last decades, some pilot urinary marker tests have become available. (Tetu 0000) These molecular tests were developed with the aim to complement cytology by detecting soluble cancer specific biomarkers. (Onal et al. 2015) However, so far, none of these point of care tests has been incorporated into clinical guidelines because their added value for the diagnosis of urothelial tumours is yet to be identified. (Schmitz-Dräger et al. 2015) Overall, no current non-invasive test has the sensitivity and specificity necessary to replace cystoscopy and so the quest for a patient friendly alternatives remains open. (Cheung et al. 2013)

The device of the first aspect is used for selective capture of target bladder cancer cells from urine. The wall of the bladder has several layers, which are made up of different types of cells. Most bladder cancers start in the innermost lining of the bladder, that is the urothelium or transitional epithelium. As the cancer grows into or through the other layers in the bladder wall, it becomes more advanced and can be harder to treat. Over time, the cancer might grow outside the bladder and into nearby structures. Therefore, devices and methods for the selective and sensitive cancer cell capture from urine are important.

Urothelial carcinoma, also known as transitional cell carcinoma (TCC), is by far the most common type of bladder cancer. These cancers start in the urothelial cells that line the inside of the bladder. Urothelial cells also line other parts of the urinary tract, such as the part of the kidney that connects to the ureter, the ureters, and the urethra. Several other types of cancer can start in the bladder, including squamous cell carcinoma, adenocarcinoma, small cell carcinoma, and sarcoma. As used herein, the term “bladder cancer”, and similar terms, means any one or more of carcinoma, squamous cell carcinoma, adenocarcinoma, small cell carcinoma, and sarcoma. The device of the first aspect can be configured to capture any one or more of these bladder cancer cells from urine.

The device of the first aspect comprises a substrate. Any suitable substrate can be used, provided a functionalized film can be formed on the surface thereof and can be retained on the surface thereof under typical operating conditions. Suitable substrate materials include glass, silicon, ceramics, metals, plastics, polymeric materials, paper, paper laminates, cellulose, carbon fibre, biomaterials, surfaces comprising biological molecules, surfaces comprising small organic molecules, surfaces comprising inorganic molecules, etc. The plastic may be selected from the group consisting of: polycarbonate, polyethylene, polypropylene, polystyrene, polytetrafluoroethylene, polyethylene terephthalate; polyethylene naphthalene dicarboxylate, tetrafluoroethylene-hexafluoropropylene copolymers, polyvinyl-difluoride, nylon, polyvinylchloride, copolymers of the aforementioned, and mixtures of the aforementioned. In embodiments, the substrate is glass. In other embodiments, the substrate is silicon.

The substrate has one or more cell capture surfaces. The one or more cell capture surfaces can be formed in one or more features on a surface of the substrate. The one or more features may be in the form of a well, such as in a 96 well plate, or they may be one or more fluid flow paths of any size, geometry or configuration. The one or more fluid flow paths may be in the form of one or more channels (open or enclosed) such as channels commonly used in “flow through” type diagnostic devices. In some embodiments, the substrate contains microfluidic features, such as microfluidic channels in a microfluidic device. As used herein, the term “microfluidic”, and variants thereof, means that the chip, device, apparatus, substrate or related apparatus contains fluid control features that have at least one dimension that is sub-millimetre and, typically less than 100 μm, and greater than 1 μm. Furthermore, the term “microchannel”, and variants thereof, means a channel having at least one dimension that is sub-millimetre and, typically less than 100 μm, and greater than 1 Wm.

In certain embodiments, the device is a microfluidic device. Thus, in a second aspect, provided herein is a microfluidic device for selective capture of target bladder cancer cells from urine or a urine derived fluid, the device comprising a substrate having one or more cell capture micro-channels, each cell capture micro-channel comprising a functionalized film on a surface thereof and one or more target bladder cancer cell selective binding agents covalently bound to the functionalized film.

In certain embodiments, the micro-channel is as described in U.S. Patent Application Publication No. 2011/0294187, which is incorporated herein by reference in its entirety. Specifically, the micro-channel can be defined with three dimensional (3D) patterns. This 3D patterning allows one to affect the flow profile within the micro-channel, which in turn enhances the interaction between the flowing sample urine solution and the cell capture surface, and subsequently significantly increases the cell capture efficiency. In some embodiments, the micro-channel surface is made from poly(dimethylsiloxane) (PDMS).

The device of the first and second aspects is used for the selective capture of target bladder cancer cells from urine or a urine derived fluid. Urine is a complex mixture containing water, salts, urea, debris, proteins, cells, and, as discussed previously, this complexity presents a challenge for urine cytologists. In contrast, the selectivity of the device of the first and second aspects means that only cancer cells are immobilised, thus overcoming problems of complexity, extracellularity and cytomorphology.

As discussed in more detail later, the selectivity of the device of the first and second aspects is brought about by using one or more binding agents that selectively bind cancer cells of interest and, more specifically, selectively bind biological markers of the cancer cells of interest. As used herein, the term “selective capture” and similar terms when used in relation to the capture of target bladder cancer cells means that, from a heterologous mixture containing the target cancer cells, other cancer cells, and other cells, the target cancer cells preferentially bind so that they are retained on the substrate and the non-target cells can be washed away or otherwise removed from the surface.

The target bladder cancer cells that can be captured using the device of the first and second aspects may be selected from one or more of the group consisting of urothelial carcinoma cells, squamous cell carcinoma cells, adenocarcinoma cells, small cell carcinoma cells, and sarcoma urine cells.

In certain embodiments, the device of the first and second aspects is used for the capture of target bladder cancer cells from humans. However, as will be appreciated by those in the art, target bladder cancer cells from other organisms may be useful in animal models of disease and drug evaluation. Thus, the target bladder cancer cells may be from other mammals, including rodents (rats, mice, hamsters, guinea pigs, etc.), primates, farm animals (including sheep, goats, pigs, cows, horses, etc.) and pets, e.g., (dogs, cats, etc.).

The cell capture surface of the device of the first and second aspects comprises a functionalized film on the substrate. The functionalized film can comprise any inorganic, organic and/or biological material, molecule or mixture of molecules that can be attached to the surface of the substrate by covalent or ionic bonding and contain one or more functional groups available for covalent or ionic bonding to target bladder cancer cell selective binding agents. As described in more detail later, the target bladder cancer cell selective binding agent in many cases will be a biological molecule, such as a peptide, protein or antibody and, therefore, the one or more functional groups of the functionalized film are preferably capable of bonding to carboxylic acid groups, carboxylate groups, amino groups or amido groups on a biological molecule.

Examples of chemistries suitable for covalent attachment of the target bladder cancer cell selective binding agent. Suitable chemistries include oxazolines, epoxies, aldehydes, anhydrides, thiols, EDC/NHS related chemistries, click chemistries, isocyanates, nitriles and imines.

In certain embodiments, the functionalized film is a polymer. The polymer may be formed by classical polymerization techniques. Thus, polymers can be formed on a surface of the substrate by polymerization of suitable starting monomers or pre-polymers using suitable polymerization agents, as is known in the art.

In other certain embodiments, the functionalized film may be formed by be formed by plasma polymerization. Thus, in certain embodiments, the functionalized film is a plasma polymer formed by plasma polymerization of one or more functional starting materials. Oxazoline, epoxy, aldehyde, anhydride, thiol, isocyanate, nitrile and imine containing starting materials can be plasma polymerized using the conditions described herein to form plasma polymers containing oxazoline, epoxy, aldehyde, anhydride, thiol, isocyanate, nitrile and imine groups that can then be reacted with the target bladder cancer cell selective binding agent to form covalent bonds therewith.

The conditions required to polymerise the one or more functional starting materials to form the plasma polymerised functionalized film may comprise a power of from about 10 W to about 50 W, a deposition time of from about 1 minute to about 7 minutes, and/or a monomer pressure of from about 1.1 to about 3×10⁻¹ mbar. A range of other deposition conditions would be applicable depending on the plasma deposition equipment design and power coupling efficacy.

Non-limiting examples of starting materials that can be used include 2-substituted oxazolines, 4-substituted oxazolines, 5-substituted oxazolines, 2,4-disubstituted oxazolines, 2,5-disubstituted oxazolines, 4,5-disubstituted oxazolines, 2,4,5-trisubstituted oxazolines, propionaldehyde (i.e. propanal), glycidyl methacrylate, and ally glycidyl ether.

In certain embodiments, the functionalized film is a plasma polymerized polyoxazoline (“PPOx”). As used herein, the term “polyoxazoline” means a homopolymer or copolymer formed from at least one oxazoline starting material or monomer. The polyoxazoline polymer may or may not comprise intact oxazoline moieties. The polyoxazoline polymer may be a copolymer formed by plasma polymerisation of at least one oxazoline starting material or monomer and at least one comonomer. The comonomer may be chosen based on the desired properties it may provide to the polyoxazoline polymer and/or its suitability for plasma polymerisation (e.g. its vapour pressure or volatility). The comonomer may be selected from the group consisting of but not limited to: silanes, siloxanes, fluorocarbons, hydrocarbons, reactive functional monomers, organo-based monomers, and unsaturated monomers such as N-vinylpyrrolidone, hydroxyethylmethacrylate, acrylamide, dimethylacrylamide, dimethylaminoethylmethacrylate, acrylic acid, methacrylic acid, a vinyl substituted polyethylene or polypropylene glycol, a vinylpyridine, and a vinylsulfonic acid.

The plasma polymerised polyoxazoline polymer and functionalised film on the surface of the substrate can be prepared by exposing the surface of a substrate to a plasma comprising an oxazoline monomer vapour under conditions to polymerise the oxazoline monomer to form the plasma polymerised polyoxazoline polymer on the surface of the substrate.

The conditions required to polymerise the oxazoline monomer to form the plasma polymerised polyoxazoline polymer may comprise a power of from about 10 W to about 50 W, a deposition time of from about 1 minute to about 7 minutes, and/or a monomer pressure of from about 1.1 to about 3×10⁻¹ mbar. A power of greater than 30 W for a time of greater than 5 minutes are particularly suitable conditions to polymerise the oxazoline monomer to form the plasma polymerised polyoxazoline polymer because they provide stable plasma polymerised polyoxazoline polymer films having a thickness of greater than about 30 nm. However, coatings with thickness above 1 nm and deposited with a range of other conditions may be suitable too.

The plasma comprising an oxazoline monomer vapour is formed at reduced pressure in a vacuum chamber. Thus, the step of exposing the surface of a substrate to a plasma comprising an oxazoline monomer vapour may include placing the substrate in a chamber, sealing the chamber, forming a plasma in the chamber, introducing a vapour containing the oxazoline monomer into the chamber, and maintaining the substrate at a temperature suitable for polymerisation of the oxazoline monomer so as to form a polymer film on the surface. Persons skilled in the art will understand that a plasma is an electrically-excited ionised gas or gases, that, upon excitation (eg. ignition), forms a highly reactive environment that can modify materials directly exposed to the plasma discharge. The plasma deposition step can be operated over a wide range of pressures (for example, from 10 mTorr to above atmospheric pressure (eg. 10× atmosphere or higher)). The plasma may consist of a combination of an inert gas (eg. helium, neon, argon, krypton, xenon, radon) and the oxazoline monomer (or other suitable monomer as required depending on the chemistry of functionalized film). The plasma can be formed at a range of frequencies (low-frequency direct current (DC) and alternating current (AC), pulsed DC, radio frequency (RF), and microwave).

The above plasma polymerization conditions can also be used to form plasma polymers from other functional starting materials as required.

The oxazoline monomer may be a substituted oxazoline with a substituent at any of the 2-, 4- or 5-positions of the oxazoline ring or any combination of these substituents. Any of these oxazolines can be used to form the plasma polymerised polyoxazoline polymer provided they are a vapour under the plasma deposition conditions used. In embodiments, the oxazoline monomer is selected from the group consisting of 2-substituted oxazolines, 4-substituted oxazolines, 5-substituted oxazolines, 2,4-disubstituted oxazolines, 2,5-disubstituted oxazolines, 4,5-disubstituted oxazolines, and 2,4,5-trisubstituted oxazolines. The substituent(s) on the oxazoline ring may be selected from the group consisting of: halogen, OH, NO₂, CN, NH₂, optionally substituted C₁-C₁₂alkyl, optionally substituted C₂-C₁₂alkenyl, optionally substituted C₂-C₁₂alkynyl, optionally substituted C₂-C₁₂heteroalkyl, optionally substituted C₃-C₁₂cycloalkyl, optionally substituted C₂-C₁₂heterocycloalkyl, optionally substituted C₂-C₁₂heterocycloalkenyl, optionally substituted C₆-C₁₈ aryl, optionally substituted C₁-C₁₈ heteroaryl, optionally substituted C₁-C₁₂alkyloxy, optionally substituted C₂-C₁₂alkenyloxy, optionally substituted C₂-C₁₂alkynyloxy, optionally substituted C₂-C₁₂heteroalkyloxy, optionally substituted C₃-C₁₂cycloalkyloxy, optionally substituted C₃-C₁₂cycloalkenyloxy, optionally substituted C₁-C₁₂heterocycloalkyloxy, optionally substituted C₁-C₁₂heterocycloalkenyloxy, optionally substituted C₆-C₁₈aryloxy, optionally substituted C₂-C₁₈heteroaryloxy, optionally substituted C₁-C₁₂alkylamino, SO₃H, SO₂NH₂, SO₂R, SONH₂, SOR, COR, COOH, COOR, CON RR, NRCOR′, NRCOOR′, NRSO₂R′, N RCON R′R″, and NRR′. In some embodiments, the oxazoline monomer comprises a 2-substituted oxazoline. In specific embodiments, the oxazoline monomer is a 2-alkyl-2-oxazoline. The alkyl substituent may be a C₁-C₁₂ alkyl, such as methyl, ethyl, n-propyl, i-propyl, n-butyl, i-butyl, t-butyl, pentyl, etc.

In specific embodiments, the oxazoline monomer is selected from the group consisting of 2-alkyl-2-oxazolines and 2-aryl-2-oxazolines. The alkyl substituent of the 2-alkyl-2-oxazolines may be a C₁-C₁, alkyl, such as methyl, ethyl, n-propyl, i-propyl, n-butyl, i-butyl, t-butyl, pentyl, and the like. The alkyl substituent may be optionally substituted. The aryl substituent of the 2-aryl-2-oxazolines may be a C₅-C₁₀, aryl, such as optionally substituted phenyl, optionally substituted naphthyl, optionally substituted thienyl, optionally substituted indolyl, and the like.

The surface of the substrate may be treated prior to deposition of the plasma polymerised polyoxazoline polymer. For example, the surface may be treated by cleaning with a detergent, water or a suitable solvent. Alternatively, or in addition, the surface may be treated by exposing the surface to air in a plasma chamber in order to activate the surface.

The plasma polymerised functionalized film may have a thickness of greater than 30 nm, such as a thickness of about 30 nm, about 40 nm, about 50 nm, about 60 nm, about 70 nm, about 80 nm, about 90 nm or about 100 nm.

The reactivity of the oxazoline ring present on the co-terminus of polyoxazolines has been used for conjugation with protein and drugs in solution. The reactivity of the oxazoline ring is believed to lead to the formation of a covalent amide bond by reaction with carboxylic acid functional groups. Plasma deposited coatings allow for the retention of intact oxazoline rings at the surface, which is typically not the case when other techniques for surface preparation are used. Retention of such reactive chemical functionalities then allows convenient and rapid covalent coupling of proteins, antibodies and the like.

The target bladder cancer cell selective binding agent can be any molecule that selective binds the target bladder cancer cells. The target bladder cancer cell selective binding agent may be a biomolecule. The biomolecule may for example be selected from amino acids, peptides, proteins, aptamers, nucleic acids, DNA molecules, RNA molecules, antibodies, growth factors, antimicrobial agents, antithrombogenic agents, and cell attachment proteins. The biomolecule may be in the form of particles or nanoparticles comprising the biomolecule.

The target bladder cancer cell selective binding agent may be attached to the functionalized film by direct reaction between one or more functional groups on the functionalized film and the target bladder cancer cell selective binding agent. For example, amino groups in the functionalized film may react with carboxylic, carboxylate or aldehyde groups of the target bladder cancer cell selective binding agent. If required, reaction between the one or more functional groups on the functionalized film and the target bladder cancer cell selective binding agent may be facilitated or catalyzed. For example, reaction between the one or more functional groups on the functionalized film and the target bladder cancer cell selective binding agent may be facilitated by a coupling agent such as a carbodiimide coupling agent (for example EDC, DCC, DIC), a triaminophosphonium coupling agent (for example BOP, PyBOP, PyBrOP) or a tetramethylaminium/tetramethyluronium coupling agent (for example HATU, HBTU, HCTU).

A cross linker moiety may be used between the functionalized film and the target bladder cancer cell selective binding agent.

The target bladder cancer cell selective binding agent can be any inorganic, organic and/or biomolecule that allows the device to selectively capture the target bladder cancer cells. The target bladder cancer cell selective binding agent may comprise at least two functional groups. A first functional group of the target bladder cancer cell selective binding agent is a moiety capable of attaching to one or more functional groups of the functionalise film, as described earlier.

Selection of the appropriate target bladder cancer cell selective binding agent is a matter of choice that depends upon the particular target bladder cancer cell population of interest.

A second functional group of the target bladder cancer cell selective binding agent is capable of binding to a cell. The binding between the second functional group and the cell can be direct or indirect. For binding directly to a cell, the second functional group itself comprises an active group capable of recognising and capturing a cell. The active groups can be specifically selected to recognise and capture a specific cell type of interest, and cell recognition and capture can be accomplished by any means known in the art. For example, cell recognition can be based on chemical or biological reactions, including without limitation peptide recognition, nucleic acid recognition and/or chemical recognition. Cell recognition can also be based on non-chemical or non-biological reaction, such as, without limitations, electrokinetic recognition or size-dependent sorting.

To indirectly bind to a cell, the second functional group of the target bladder cancer cell selective binding agent is attached to a cell through a separate cell-binding agent. Such cell-binding agents are well-known in the art. The cell-binding agent may be a single component or be in a form of complex comprising two or more components, as long as at least one of the components is capable of binding to a target cell. For example, in addition to the component directly binding to a cell, the cell-binding agent or complex may comprise an additional component attached to the component binding to the cell.

For example, the target bladder cancer cell selective binding agent, or one component of the target bladder cancer cell selective binding agent, can be an antibody, a lymphokine, a hormone, a growth factor, or any other cell-binding molecule or substance that specifically binds a target bladder cancer cell.

In certain embodiments, the target bladder cancer cell selective binding agent is one or more antibodies, or fragments thereof. The antibodies may be selected from: polyclonal antibodies or monoclonal antibodies, including fully human antibodies; single chain antibodies (polyclonal and monoclonal); fragments of antibodies (polyclonal and monoclonal) such as Fab, Fab′, F(ab′)₂, and Fv; chimeric antibodies and antigen-binding fragments thereof, and domain antibodies (dAbs) and antigen-binding fragments thereof, including camelid antibodies. In certain specific embodiments, the target bladder cancer cell selective binding agent is a monoclonal antibody. In other certain specific embodiments, the target bladder cancer cell selective binding agent is a polyclonal antibody.

Monoclonal antibody techniques allow for the production of specific cell-binding agents in the form of monoclonal antibodies. Techniques for creating monoclonal antibodies are well known in the art. Such antibodies can be produced by, for example, immunizing mice, rats, hamsters or any other mammal with the antigen of interest. Antigens of interest may include the intact target bladder cancer cell, antigens isolated from the target bladder cancer cell, whole virus, attenuated whole virus, or viral proteins such as viral coat proteins. Sensitized human cells can also be used. Another method of creating monoclonal antibodies is the use of phage libraries of scFv (single chain variable region), specifically human scFv.

The target bladder cancer cell selective binding agent can also be a combination of two or more different kind antibodies.

Thus, the device of the first and second aspects may comprise an immobilized functional antibody capable of selective capture of target balder cancer cells. In certain specific embodiments, the antibody is anti-Epithelial Cell Adhesion Molecule (anti-EpCAM), which specifically binds to EpCAM expressing cancer cells in urine. Urothelial carcinoma is such an EpCAM expressing cancer cell type. Other EpCAM expressing cancer cell types include those that correspond to HT1197, HT1376, RT4 and EJ 138 bladder cancer cell lines. By way of example, HTC116 cells are a carcinoma cell type with a particularly high expression level of EpCAM. Other antibodies that could be used include E-cadherin, CA19-9, CD146, CD147, CD10, CD44, CD24, CD133, CD166, mucins (for instance MUC1 and MUC4), cadherins (CDH 1-28), uroplakins, and Lewis antigen antibodies.

One important challenge is for the antibody to remain strongly bound to the cell capture surface despite high variability in pH and ionic concentration in urine samples. This challenge is overcome by using the polyoxazoline plasma polymers described earlier to covalently bind the antibody to the substrate. In the examples provided herein, the substrate was optimised stepwise for the isolation of cancer cells spiked in biologically relevant media from physiological buffer through artificial urine and ultimately real patient urine. The selectivity of the device for cancer cells was tested against podocyte cells, a kidney epithelial cell which can also occur in the urine of diseased patients. (Sir Elkhatim et al. 2014)

Non-antibody molecules can also be used to target specific target bladder cancer cell populations.

Once captured on the cell capture surface of the substrate, target bladder cancer cells can be separated from urine and other components of urine by washing, for example with PBS.

Captured target bladder cancer cells can be detected and/or analysed using any of several methods known to those skilled in the art. Captured target bladder cancer cells can be observed using a photomicroscope. Captured target bladder cancer cells may be detected by fluorescent or luminescent labelling. For example, captured target bladder cancer cells can be imaged using fluorescent microscopy. The number of cells bound to the surface can then be computed.

Advantageously, captured bladder cancer cells can be differentiated in vitro from healthy cells in using a cancer specific fluorescently active compound. For example, ALA 5 is a compound that is metabolized quicker by cancer cells leading to emission of light which can be used in identifying captured cancer cells. Other cancer specific fluorescently active compounds that can be used include hexaminolevulinate and hypericin.

If desired, captured target bladder cancer cells can be selectively released from the capture surface of the substrate.

In a third aspect, the present disclosure provides a method of selective capturing target bladder cancer cells from urine or a urine derived fluid, the method comprising:

providing a sample of urine or a urine derived fluid;

providing a substrate having one or more cell capture surfaces, each cell capture surface comprising a functionalized film on the substrate and one or more target bladder cancer cell selective binding agents covalently bound to the functionalized film;

contacting the sample of urine or a urine derived fluid with the one or more cell capture surfaces under conditions to bind at least some of the target bladder cancer cells from the urine (if present) to the cell capture surface.

The method of the third aspect may further comprise detecting target bladder cancer cells on the one or more cell capture surfaces.

In a fourth aspect, the present disclosure provides a method of immobilising target bladder cancer cells from urine or a urine derived fluid on a substrate surface, the method comprising:

providing a sample of urine or a urine derived fluid;

providing a substrate having one or more cell capture surfaces, each cell capture surface comprising a functionalized film on the substrate and one or more target bladder cancer cell selective binding agents covalently bound to the functionalized film;

contacting the sample of urine or a urine derived fluid with the one or more cell capture surfaces under conditions to bind at least some of the target bladder cancer cells from the urine (if present) to the cell capture surface.

In a fifth aspect, the present disclosure provides a method for diagnosing or monitoring bladder cancer in a mammal, the method comprising:

providing a sample of urine or a urine derived fluid obtained from the mammal;

providing a substrate having one or more cell capture surfaces, each cell capture surface comprising a functionalized film on the substrate and one or more target bladder cancer cell selective binding agents covalently bound to the functionalized film;

contacting the sample of urine or a urine derived fluid with the one or more cell capture surfaces under conditions to bind at least some of the target bladder cancer cells from the urine (if present) to the cell capture surface; and

analyzing the target bladder cancer cells bound to the cell capture surface.

The devices and methods described herein provide a rapid and selective method for capture of target bladder cancer cells from urine. Current urinary diagnostic tests for bladder cancer are expensive and have limited sensitivity and specificity. The devices and methods described herein provide the first generation of specific urinary tests for the detection of cancer cells in urine. The unique reactivity of plasma deposited polyoxazoline was used to covalently bind cancer specific antibodies in micro-channels. Cancer cells dispersed in patient urine were successfully captured with up to 99% selectivity and 100% sensitivity over a wide range of cell concentrations. The streamlined two steps preparation process of the capture platform represents an important advance in medical diagnostics.

### EXAMPLES

Materials

Specific supplies consisted of polyclonal goat anti-human EpCAM affinity purified polyclonal antibody (bio scientific, AF960), Alexa fluor 630 donkey anti-goat secondary anti-body (Novex), RPMI1640 media, McCoy's 5A modified media, ibidi channel sticky-Slide VI 0.4 and celltracker dyes (Oregon green and CMPTX red, life technology). Phosphate buffer saline (PBS) solution was purchased from Sigma. Substrate blocking media consisted of PBS with 0.01 w % skim milk powder. Artificial urine solution consisted of 0.17M NaCl, 0.08M KCl, 0.52M NaH₄PO₄ and 700 mg/L urea, all reagents were purchased from Sigma. (Haddad 2006) Native urine specimen (10 mL, morning void) were obtained from healthy donors. Ethical approval number 442.13/HREC/13/SAC/283 was obtained to collect clinical specimen from the Southern Adelaide Clinical Human Research Ethics Committee (SAC HREC EC00188).

### Example 1—Preparation of a 96 Well Plate Plasma Polymer Substrate

For cell capture, 96 well plates (Corning, Costar) and microscopy grade glass slides (Objekttrager) were used as solid substrates. Silicon wafers were used for plasma polymer film thickness measurements.

Prior to plasma deposition, glass slides and silicon wafers were washed in piranha solution (H₂O₂:H₂SO₄, 1:3) and rinsed extensively with milliQ water. Sterile 96 well plates were used as received.

An oxazoline based thin film coating was deposited onto the solid substrate via continuous mode plasma deposition as described previously. (Macgregor-Ramiasa et al. 2015b; Ramiasa et al. 2015) Briefly, a tailor-made parallel plate plasma reactor was brought under vacuum (2·10⁻² mbar), and 2-methyl-2-oxazoline precursor (Sigma-Aldrich, Australia) inoculated in the chamber with a needle valve until a constant monomer flow rate of 8 standard cubic centimetre per minute (sccm) was reached. The plasma was then ignited with a continuous radio frequency power of 50 W and the film deposited for 3 minutes. The thin films of plasma deposited polyoxazoline (PPOx) obtained in this way were kept in a desiccator for no longer than 1 week prior to further characterisation or functionalization.

Physico-Chemical Analysis of Plasma Deposited Polyoxazoline Film

The thickness of the PPOx films was measured on silicon wafers by ellipsometry (Variable Angle Spectroscopic Ellipsometer, J.A Woolam Co. Inc., USA) after incubation in MilliQ water, phosphate buffer saline (PBS), artificial and native urine. The atomic composition of the plasma deposited polyoxazoline thin films was determined by X-ray photoelectron spectroscopy (SPECS SAGE, Germany, monochromatic Mg Kα radiation source, 15 KV, 10 mA). Survey spectra were acquired between 0 and 1100 eV, at a pass energy of 120 eV with a resolution of 1 eV. Time of flight Secondary Ion Mass Spectroscopy (PHI TRIFT V nano ToF, Physical Electronics, USA) was used to characterise the chemistry of the plasma deposited polyoxazoline thin films.

### Example 2—Preparation of a Microfluidic Plasma Polymer Substrate

A PPOx coated microscope glass slide was formed using the method described in Example 1. Microchannels were then formed between the PPOx coated microscope glass slide and an Ibidi IV 0.4 sticky Slide® (DSKH) using standard techniques.

### Example 3—Covalent Attachment of Antibody to Plasma Polymer Substrate

The cell capture “chambers” consisted of either the individual wells of a standard 96-well plate (Example 1) or microchannels (Example 2). Antibodies were irreversibly bound to the plasma polymer substrate using the unique reactivity of plasma deposited polyoxazoline with carboxylic acid (Macgregor-Ramiasa et al. 2015a; Schmidt et al. 1994; Tillet et al. 2011). Anti-EpCAM antibodies were dissolved in 100% PBS at 10 μg·mL₋₁ and 50 μL gently pipetted onto the plasma polymer substrate and allowed to bind overnight at 4° C. The antibody solution was then aspirated and the active polymer surface blocked with skim milk by incubating for 15 min in a 0.1 mg·mL⁻¹ solution. The substrate was then thoroughly rinsed with PBS. The immobilisation of the antibody onto the PPOx substrate was confirmed with ToF SIMS analysis.

### Example 4—EpCAM Marker is Overexpressed in Cancer Cells

Three independent methods were used to demonstrate that EpCAM and other antibodies such as E-cadherin are over expressed on various bladder cancer cell lines, as shown in FIG. 2.

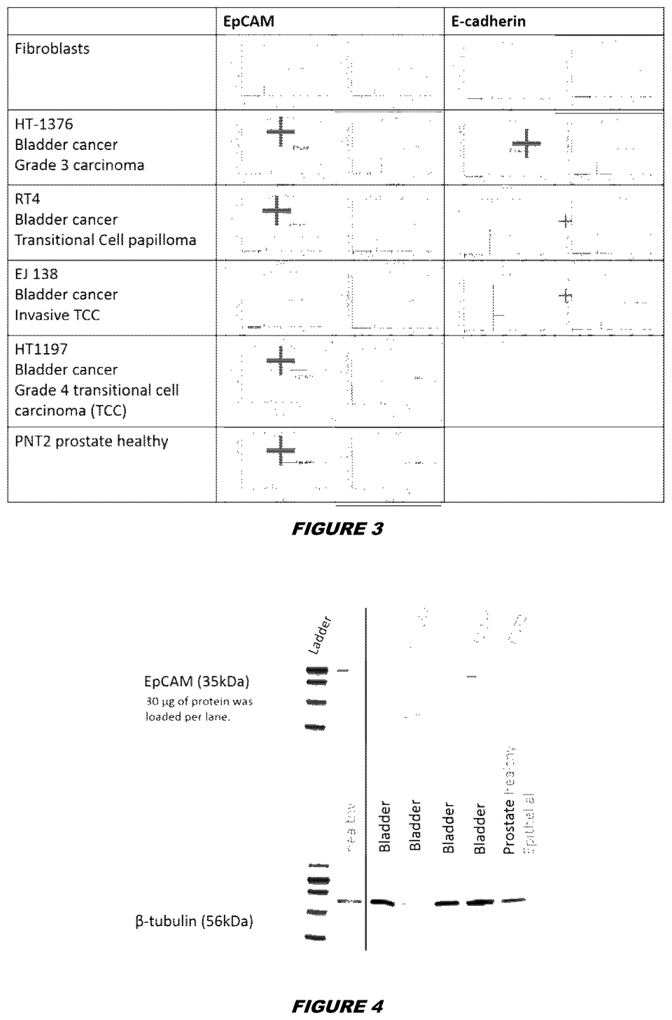

The bladder cancer cell lines investigated were bladder cancer HT1197, HT1376, RT4 and EJ 138. These cell lines correspond to different cancer grades as summarised in the whole FACS data set table shown in FIG. 3. The control cell lines were fibroblasts and healthy epithelial PNT2 prostate cells. Full data sets for western blots, FACS and immunostaining are shown in FIG. 3.

The western blots (FIG. 4) confirm the FACS EpCAM expression results: control fibroblasts are EpCAM negative; cancer cells are EpCAM positive, except EJ138; healthy epithelial PNT2 are EpCAM positive.

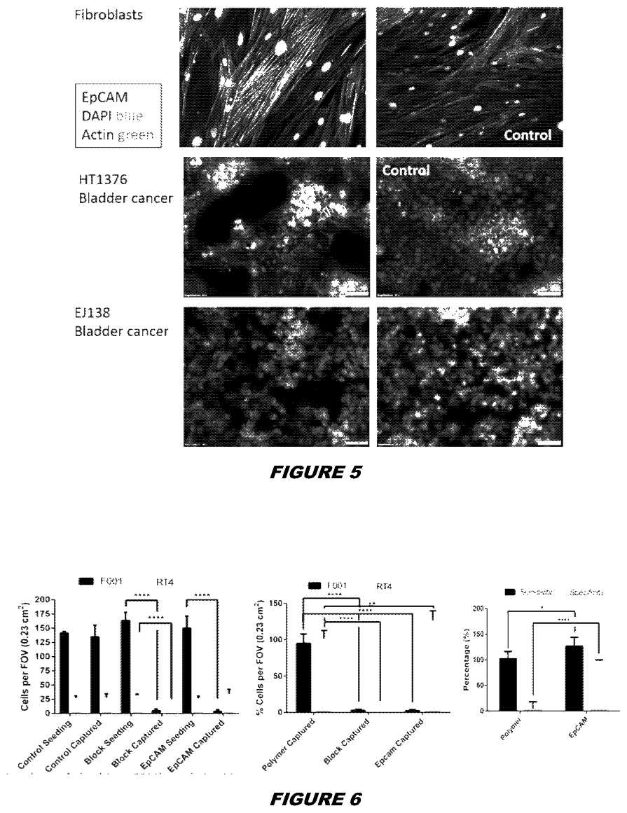

The immunostaining results (FIG. 5) confirm the FACS and western blots: control fibroblasts are EpCAM negative; cancer cells HT1376 are EpCAM positive, not EJ138.

### Example 5—Selective Capture of Spiked Cancer Cells from Urine

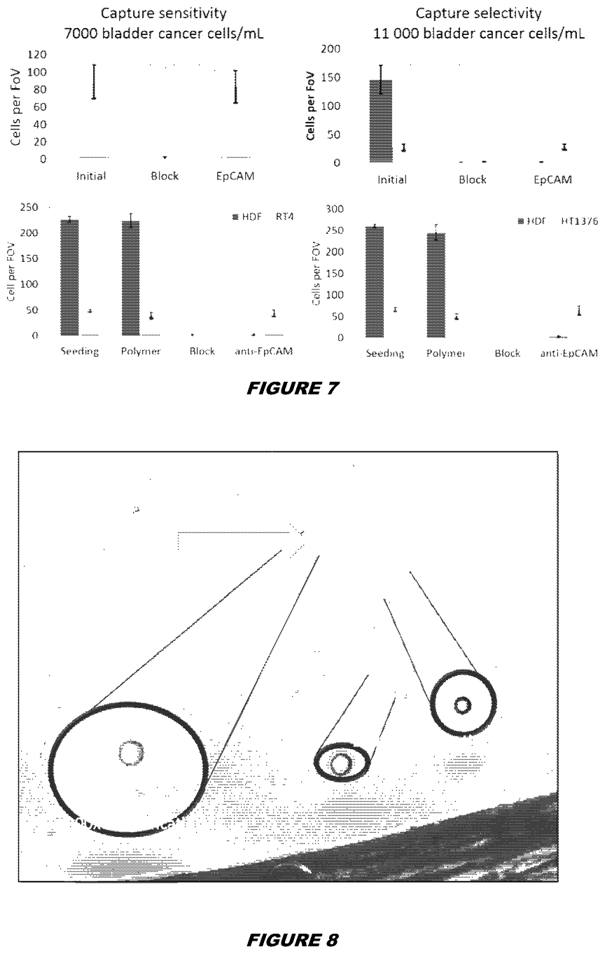

We demonstrated that overexpression of EpCAM in bladder cancer cell lines allows their capture into functionalised fluidics channels. We have shown that RT4 cells can be captured (FIG. 6). We have also demonstrated that other bladder cancer cell lines, for instance HT1376, are selectively and sensitively captured (FIG. 7).

Bladder cancer cell lines (RT4, HT1376 or HT1197) and healthy control cells (F001) fibroblasts were stained separately prior to the capture experiment with red (celltracker CMPTX) and green (CellTrace Oregon green) fluorescent dyes. Once fluorescently labelled, the cells were spiked into real healthy urine samples in various absolute numbers and ratio. 50 uL of the spiked urine samples were introduced in the EpCAM functionalised and control microchannels. The surface of the negative control microchannel was blocked with skim milk protein to prohibit cell adsorption. The surface of the positive control channels consisted of a biocompatible plasma deposited polyoxazoline coating, which promotes non-specific cell binding. After a 15 to 30 minute incubation the urine was aspirated and the channels rinsed with PBS buffer. The number of cells bound in all three type of channels was determined from a minimum of four low magnification fluorescent images of the channel surface, capture with both red and green filters.

### Example 6—Bladder Cancer Cells can be Selectively and Sensitively Captured

Experiments were also conducted under flow conditions in which bladder cancer cell lines spiked in low number into healthy patient urine were captured in EpCAM functionalised PPOx fluidics channels. FIG. 8 shows cancer cells captured under the following flow conditions:
1000 HT1376 bladder cancer cells spiked in 5 mL of urine;
cells flowed through channel at a flow rate of 6 uL/min; and
PBS buffer rinse at 10 uL/min.

The results show that targeted cells can be captured under flow conditions. This shows that the surface-immobilised antibody is still bound and functional in these extreme conditions.

### Example 7—Capturing Cancer Cells from Real Patient Urine Samples

While in spiked cell experiments it was easy to artificially stain in red or green the cancer and healthy cells separately, in real cancer patients a new way was required to confirm that the cells captured are indeed cancerous.

We used an approach based on existing protocols used in cystoscopy to enhance visual demarcation between cancerous and healthy cells.

Cancer specific fluorescence is difficult to use at the scale of single cells (as opposed to whole tumors) and in vitro (as opposed to in vivo for cystoscopy) because the induce fluorescence is feeble and bleaches easily.

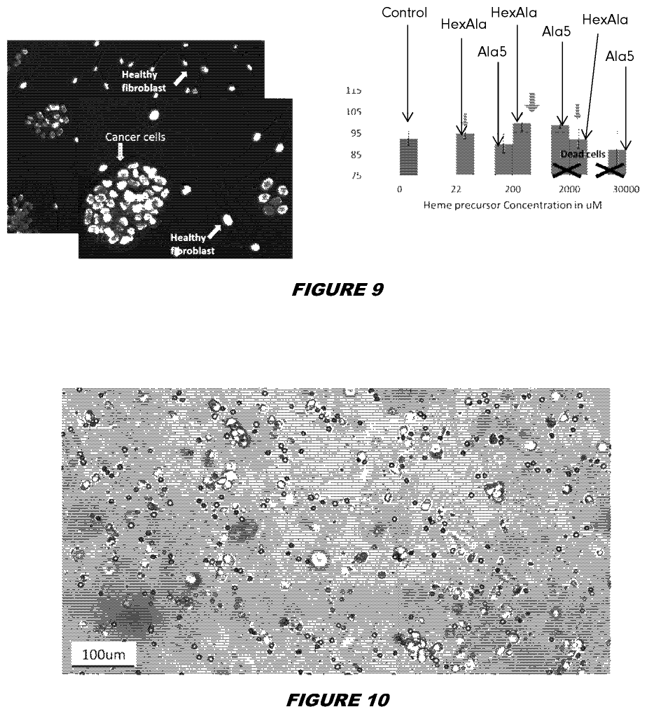

Nonetheless, we used three different cancer specific fluorescently active compounds (5-ALA, its derivative hexaminolevulinate and hypericin) to differentiate in vitro, cancer cell lines from healthy cells in co-culture, based on their distinctive fluorescence (FIG. 9).

An example of the cellularity of real cancer patient samples is shown in FIG. 10. Clumps of suspicious cancer cells clearly display the specific red fluorescence.

Capture experiments were conducted in the same conditions as for the spiked cells experiments: a blocked channel and a plain PPOx channel were used as negative and positive control respectively. The test channel consisted of EpCAM functionalised PPOx channel. Hex ALA was added to the urine samples prior to testing, together with DAPI nuclear stain in order to be able to distinguish between cells and other metabolites.

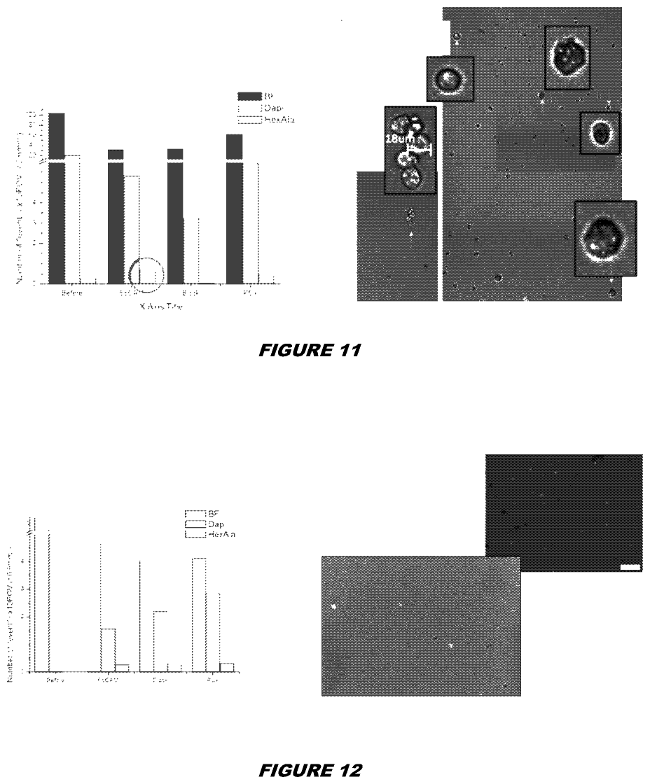

Results for one patient sample with confirmed bladder cancer are shown in FIG. 11.

All cells were counted before and after rinsing the channels, we distinguished between “bright field event”, grey bars labelled BF (=any kind of bound cell but also metabolite of other sorts), cells on the blue bars labelled DAPI (=distinctive blue dapi stain) and “suspicious” cells with red bars labelled Hexala. The latter correspond to entities that both have a nucleus and red fluorescence. Cells with features distinctive of cancer cells (large nucleus, irregular shape, distinctive red fluorescence) were captured in the EpCAM channels, in much larger proportion than in the block channel: ratio EpCAM/block was 12.6.

An example of a known healthy sample is shown in FIG. 12. The sample cellularity is in this case much less and there is no significant difference between the numbers of suspicious cells captured on block or EpCAM channels, both in very low numbers. The EpCAM/block ratio was here less than 1.

### Example 8—Cancer Cells can be Captured on EpCAM Functionalised Plasma Polymers from Real Patient Samples and the Identity of these Cells can be Confirmed with Ala-5 Induced Fluorescence

More patients were analysed, and the results of the tests compared to the results of cytology and cystoscopy, which is considered as the gold standard in terms of bladder cancer diagnostics.

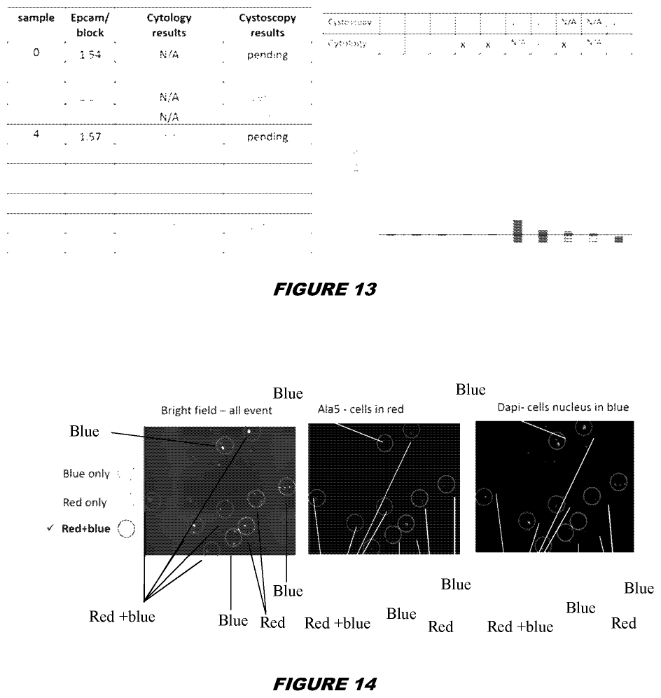

The EpCAM/block ratio found in our experiment correlated well with the finding of cystoscopy and a threshold can be determined to surpass cytology results (FIG. 13).

Samples of three confirmed healthy samples were tested for which our test systematically returned low EpCAM/block ratio values, below 3. The samples of five patients with confirmed bladder cancer were tested. Amongst those five, two were considered positive for cancer by both cystoscopy and cytology. Those two samples were the ones for which our test returned the highest EpCAM/block ratio, above 12. 2 were considered by cystoscopy as low grade cancers, but negative according to cytology. One was labelled “tumor” by cystoscopy but just highly suspicious by cytology. For the last three samples our test returned intermediate EpCAM/block values between 4 and 7.

Based on the results collected from these patient samples, using a threshold value of three, our test is capable of distinguishing between healthy and low grade cancers.

### Example 9—Automated Counting Software

Software can be used to identify the captured cancer cells automatically based on cell fluorescence color (red+blue), fluorescence intensity, shape, and size selection criteria (FIG. 14). Traditionally, cancer cells are identified via cytology due to the irregular and large nucleus, higher nuclear to cytoplasm ratio, darker (more DNA in the nucleus), smaller then Granulomas, polyoma virus, casts but bigger than bacteria, RBC. In contract, cells can be identified in the device disclosed herein because the nucleus of cancer cells appear blue, cells that are Ala5 positive appear red and the cancer cells generally have a size of greater than 10 microns and less than 30 nm.

### Example 10—Optimal Conditions for the Cancer Specific Photosensitizer

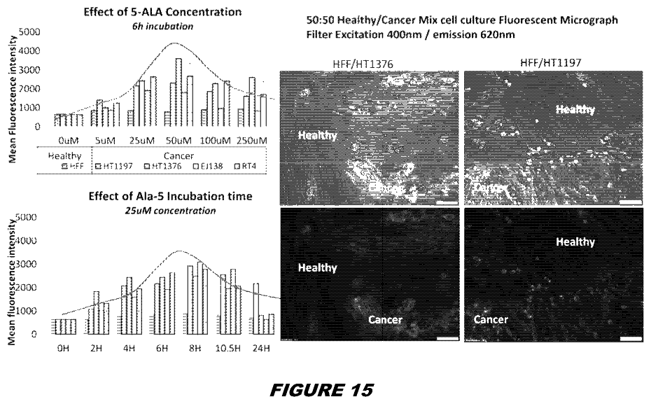

The optimal conditions for the cancer specific photosensitizer ex-vivo were investigated. Specifically, the concentration ranges for Hexaminulevulinic acid (HexAla-5) were tested for different incubation times. The results are provided in FIG. 15 which shows that the difference between the mean fluorescence of healthy (HFF) cells and four different cancer cell lines is clear for HexAla concentrations between 5 and 100 mM (top left), and incubation times between 2 and 8 h (bottom left). Images for the ideal conditions with cancer cell lines HT1376 and HT1197 in 50:50 co-culture with healthy human foreskin fibroblasts (HFF) are shown in the fluorescent micrographs on the right. The top images are an overlay with the bright field image to see the non-fluorescent healthy cells present.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

### REFERENCES

Andersson, E., Steven, K., Guldberg, P., 2014. PLoS One 9(4), e94023.
Arya, S. K., Lim, B., Rahman, A. R. A., 2013. Lab on a Chip 13(11), 1995-2027.
Bastacky, S., Ibrahim, S., Wilczynski, S. P., Murphy, W. M., 1999. Cancer Cytopathol. 87(3), 118-128.
Brimo, F., Vollmer, R. T., Case, B., Aprikian, A., Kassouf, W., Auger, M., 2009. Am. J. Clin. Pathol. 132(5), 785-793.
Bryan, R. T., Kirby, R., O'Brien, T., Mostafid, H., 2014a. Eur. Urol. 66(2), 263-264.
Bryan, R. T., Shimwell, N. J., Wei, W., Devall, A. J., Pirrie, S. J., James, N. D., Zeegers, M. P., Cheng, K. K., Martin, A., Ward, D. G., 2014b. Br. J. Cancer 110(3), 679-685.
Cavallaro, A. A., Macgregor-Ramiasa, M. N., Vasilev, K., 2016. ACS Applied Materials & Interfaces 8(10), 6354-6362.
Cheung, G., Sahai, A., Billia, M., Dasgupta, P., Khan, M. S., 2013. BMC Med. 11(1), 1-8.
Dawam, D., 2012. In: Canda, D. A. (Ed.), Bladder Cancer—From Basic Science to Robotic Surgery. INTECH Open Access Publisher.
Deden, C., 1954. Store Nordiske Videnskabsboghandel, Copenhagen.
Dharmasiri, U., Witek, M. A., Adams, A. A., Soper, S. A., 2010. Annual Review of Analytical Chemistry 3(1), 409-431.
Gaston, K. E., Pruthi, R. S., 2004. Urology 63(6), 1009-1016.
Gubala, V., Harris, L. F., Ricco, A. J., Tan, M. X., Williams, D. E., 2011. Anal. Chem. 84(2), 487-515.
Györy, A., Hawkins, T., Ross, M., McLennan, J., Ibels, L., 1998. Lab. Hematol. 4, 211-216.
Haddad, L., 2006. Google Patents.
Kaplan, A. L., Litwin, M. S., Chamie, K., 2014. Nature Reviews Urology 11(1), 59-62.
Kiyoshima, K., Akitake, M., Shiota, M., Takeuchi, A., Takahashi, R., Inokuchi, J., Tatsugami, K., Yokomizo, A., Eto, M., 2016. Anticancer Res. 36(2), 799-802.
Lin, C.-W., Liao, M.-Y., Lin, W.-W., Wang, Y.-P., Lu, T.-Y., Wu, H.-C., 2012. The Journal of Biological Chemistry 287(47), 39449-39459.
Lotan, Y., Kamat, A. M., Porter, M. P., Robinson, V. L., Shore, N., Jewett, M., Schelhammer, P. F., White, R. d., Quale, D., Lee, C. T., 2009. Cancer 115(18), 4096-4103.
Macgregor-Ramiasa, M. N., Cavallaro, A. A., Vasilev, K., 2015a. Journal of Materials 517 Chemistry B. 26
Macgregor-Ramiasa, M. N., Cavallaro, A. A., Vasilev, K., 2015b. Journal of Materials Chemistry B 3(30), 6327-6337.
MacGregor-Ramiasa, M. N., Cavallaro, A. A., Visalakshan, R. M., Gonzalez, L., Vasilev, K., 2016. CHEMECA 2016: Chemical Engineering—Regeneration, Recovery and Reinvention, pp. 302-3012. Engineers Australia, Adelaide, SA.
McGrew, E. A., 1961. Postgrad. Med. J. 37(430), 456-467.
Mitra, A. P., Cote, R. J., 2010. Nature reviews. Urology 7(1), 11-20.
Murphy, W. M., 1990. Hum. Pathol. 21(9), 886-896.
Murphy, W. M., Soloway, M. S., Jukkola, A. F., Crabtree, W. N., Ford, K. S., 1984. Cancer 53(7), 1555-1565.
Nagrath, S., Sequist, L. V., Maheswaran, S., Bell, D. W., Irimia, D., Ulkus, L., Smith, M. R., Kwak, E. L., Digumarthy, S., Muzikansky, A., Ryan, P., Balis, U. J., Tompkins, R. G., Haber, D. A., Toner, M., 2007. Nature 450(7173), 1235-1239.
Ni, L. A. N., Saleem, M., Mathieson, P. W., 2012. Nephrology 17(6), 525-531.
Oliveira Arcolino, F., Tort Piella, A., Papadimitriou, E., Bussolati, B., Antonie, D. J., Murray, P., van den Heuvel, L., Levtchenko, E., 2015. Stem Cells International 2015, 7.
Onal, B., Han, U., Yilmaz, S., Koybasioglu, F., Altug, U., 2015. Diagn. Cytopathol. 43(4), 307-314.
Papanicolaou, G. N., Marshall, V. F., 1945. Science 101(2629), 519-520.
Patriarca, C., Macchi, R. M., Marschner, A. K., Mellstedt, H., 2012. Cancer Treat. Rev. 38(1), 68-75.
Ploeg, M., Aben, K. K. H., Kiemeney, L. A., 2009. World J. Urol. 27(3), 289-293.
Ramiasa, M. N., Cavallaro, A. A., Mierczynska, A., Christo, S. N., Gleadle, J. M., Hayball, J. D., Vasilev, K., 2015. Chem. Commun. 51(20), 4279-4282.
Ruf, P., Gires, O., Jager, M., Fellinger, K., Atz, J., Lindhofer, H., 2007. Br. J. Cancer 97(3), 315-321.
Saleem, M. A., Ni, L., Witherden, I., Tryggvason, K., Ruotsalainen, V., Mundel, P., Mathieson, P. W., 2002a. The American Journal of Pathology 161(4), 1459-1466.
Saleem, M. A., O'Hare, M. J., Reiser, J., Coward, R. J., Inward, C. D., Farren, T., Xing, C. Y., Ni, L., Mathieson, P. W., Mundel, P., 2002b. J. Am. Soc. Nephrol. 13(3), 630-638.
Sanders, W., 1864. Edinburgh Journal of Medicine 10, 273-274.
Schmidt, D. L., Coburn, C. E., DeKoven, B. M., Potter, G. E., Meyers, G. F., Fischer, D. A., 1994. Nature 368(6466), 39-41.
Schmitz-Dräger, B. J., Droller, M., Lokeshwar, V. B., Lotan, Y., Hudson, M. A., van Rhijn, B. W., Marberger, M. J., Fradet, Y., Hemstreet, G. P., Malmstrom, P. U., Ogawa, O., Karakiewicz, P. I., Shariat, S. F., 2015. Urol. Int. 94(1), 1-24.
Sir Elkhatim, R., Li, J. Y., Yong, T. Y., Gleadle, J. M., 2014. Expert Rev Mol Diagn 14(4), 423-437.
Tetu, B., 0000. Mod. Pathol. 22(S2), S53-S59.
Tillet, G., Boutevin, B., Ameduri, B., 2011. Progress in Polymer Science (Oxford) 36(2), 191-217.
Vasilev, K., Michelmore, A., Griesser, H. J., Short, R. D., 2009. Chem. Commun. (24), 3600-3602.
Vasilev, K., Michelmore, A., Martinek, P., Chan, J., Sah, V., Griesser, H. J., Short, R. D., 2010. Plasma Processes and Polymers 7(9-10), 824-835.
Wronska, D. B., Krajewska, M., Lygina, N., Morrison, J. C., Juzumiene, D., Culp, W. D., Nair, S. A., Darby, M., Hofmann, C. M., 2014. Biotechniques 57(2), 63-71.
Yoon, H. J., Kozminsky, M., Nagrath, S., 2014. ACS Nano 8(3), 1995-2017.
Youssef, R. F., Lotan, Y., 2011. The Scientific World Journal 11, 369-381.
Zanini, S., Zoia, L., Dell'Orto, E. C., Natalello, A., Villa, A. M., Pergola, R. D., Riccardi, C., 2016. Materials & Design 108, 791-800.
Zhang, J., Zheng, S., Fan, Z., Gao, Y., Di, X., Wang, D., Xiao, Z., Li, C., An, Q., Cheng, S., 2001. Cancer Lett. 172(1), 55-58.
Zhe, X. N., Cher, M. L., Bonfil, R. D., 2011. Am. J. Cancer Res. 1(6), 740-751.
Zhu, H., Dale, P. S., Caldwell, C. W., Fan, X., 2009. Anal. Chem. 81(24), 9858-9865.

The reference to any prior art in this specification is not, and should not be taken as, an acknowledgement of any form of suggestion that such prior art forms part of the common general knowledge.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

1. A device for selective capture of target bladder cancer cells from urine or a urine derived fluid, the device comprising:
a substrate having one or more cell capture surfaces, each cell capture surface comprising a functionalized film on the substrate and one or more target bladder cancer cell selective binding agents covalently bound to the functionalized film.

2. (canceled)

3. The device of claim 1, wherein the target bladder cancer cells are selected from one or more of the group consisting of urothelial carcinoma cells, squamous cell carcinoma cells, adenocarcinoma cells, small cell carcinoma cells, and sarcoma urine cells.

4. The device of claim 3, wherein the target bladder cancer cells are urothelial carcinoma cells.

5. The device of claim 1, wherein the functionalized film is a plasma polymerized polyoxazoline.

6. The device of claim 1, wherein the target bladder cancer cell selective binding agent is an immobilized functional antibody capable of selective capture of target bladder cancer cells.

7. The device of claim 1, wherein the antibody is anti-Epithelial Cell Adhesion Molecule (anti-EpCAM).

8. A method of selective capturing or immobilizing target bladder cancer cells from urine or a urine derived fluid, the method comprising:
providing a sample of urine or a urine derived fluid;
providing a substrate having one or more cell capture surfaces, each cell capture surface comprising a functionalized film on the substrate and one or more target bladder cancer cell selective binding agents covalently bound to the functionalized film;
contacting the sample of urine or a urine derived fluid with the one or more cell capture surfaces under conditions to bind at least some of the target bladder cancer cells from the urine (if present) to the cell capture surface.

9. (canceled)

10. A method for diagnosing or monitoring bladder cancer in a mammal, the method comprising:
providing a sample of urine or a urine derived fluid obtained from the mammal;
providing a substrate having one or more cell capture surfaces, each cell capture surface comprising a functionalized film on the substrate and one or more target bladder cancer cell selective binding agents covalently bound to the functionalized film;
contacting the sample of urine or a urine derived fluid with the one or more cell capture surfaces under conditions to bind at least some of the target bladder cancer cells from the urine (if present) to the cell capture surface; and
analyzing the target bladder cancer cells bound to the cell capture surface.

11. The method of claim 8, wherein the target bladder cancer cells are selected from one or more of the group consisting of urothelial carcinoma cells, squamous cell carcinoma cells, adenocarcinoma cells, small cell carcinoma cells, and sarcoma urine cells.

12. The method of claim 11, wherein the target bladder cancer cells are urothelial carcinoma cells.

13. The method of claim 8, wherein the functionalized film is a plasma polymerized polyoxazoline.

14. The method of claim 8, wherein the target bladder cancer cell selective binding agent is an immobilized functional antibody capable of selective capture of target bladder cancer cells.

15. The method of claim 8, wherein the antibody is anti-Epithelial Cell Adhesion Molecule (anti-EpCAM).

16. (canceled)

17. (canceled)

18. The method of claim 10, wherein the target bladder cancer cells are selected from one or more of the group consisting of urothelial carcinoma cells, squamous cell carcinoma cells, adenocarcinoma cells, small cell carcinoma cells, and sarcoma urine cells.

19. The method of claim 18, wherein the target bladder cancer cells are urothelial carcinoma cells.

20. The method of claim 10, wherein the functionalized film is a plasma polymerized polyoxazoline.

21. The method of claim 10, wherein the target bladder cancer cell selective binding agent is an immobilized functional antibody capable of selective capture of target bladder cancer cells.

22. The method of claim 10, wherein the antibody is anti-Epithelial Cell Adhesion Molecule (anti-EpCAM).
